(19) United States
(12) Patent Application Publication
(10) Pub. No.: US 2005/0148518 A1
(43) Pub. Date: Jul. 7, 2005

(54) Anti-bacterial compositions

(75) Inventors: Elizabeth Carol McWilliam Leitch, Aberdeenshire (GB); Sylvia Helen Duncan, Aberdeenshire (GB); Harry James Flint, Aberdeenshire (GB); Colin Samuel Stewart, Aberdeenshire (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Rowett Research Institute, Bucksburn, Aberdeen (GB)

(21) Appl. No.: 10/502,996

(22) PCT Filed: Jan. 31, 2003
(86) PCT No.: PCT/GB03/00401

(30) Foreign Application Priority Data
Jan. 31, 2002 (GB) 020187.1

(51) Int. Cl. A61K 31/7048; A61K 31/366; A61K 31/19

(52) U.S. Cl. 514/27; 514/457; 514/557

(57) ABSTRACT

Anti-bacterial composition comprising an admixture of an organic acid (excluding acetate, propionate and butyrate) together with a coumarin or coumarin glycoside. Preferred organic acids include lactate, citrate and benzoate, especially L-lactate. Preferred coumarins are esculetin, scopoletin, imbelliferone and Coumarin (1,2-benzopyrone). The composition, which is effective against E. coli 0157, Salmonella, Listeria, Campylobacter and MRSA, can be used to disinfect buildings or instruments and in food preparation, e.g., as a vegetable wash.

The present invention relates to food safety and also to the treatment of bacterial infections, in particular due to E. coli O157 and other foodborne pathogenic bacteria.

Foodborne bacterial pathogens are a major cause of concern to public health, presenting the food industry with a severe challenge. Escherichia coli O157 is a prime example. This bacterium is the causative agent of haemorrhagic colitis and haemolytic uremic syndrome. Infections caused by E. coli O157, though infrequent, are associated with a high level of morbidity and mortality, particularly in the young and the elderly. The severity of infections caused by E. coli O157 and other pathogenic bacteria has attracted a high level of media attention. This has resulted in reduced public confidence in food safety, particularly in red meat products and products such as salad leaves and other vegetables which may be treated with fertilisers containing animal manure.

There is a growing interest in how bacterial pathogens enter the food chain and practical measures of prevention. Escherichia coli O157, for example, has a very low infectious dose and it may be carried asymptomatically by farm animals including cattle, sheep, pigs, turkeys etc. Farm animals including cattle and sheep are regarded as a primary reservoir of E. coli O157. Moreover, this organism is repeatedly isolated from the farmyard which strongly implicates this environment in the persistence of E. coli O157 (LeJeune et al. (2001)). Cattle faeces are a major source of contamination of meat products in slaughter houses, and slurry is a potential source of contamination of local water supplies and crops, as well as vegetables and fruit which maybe eaten raw. Farm workers and visitors, veterinarians and slaughterhouse staff are also at risk of infection from farm animal faeces. The practice of feed withdrawal during transport to slaughter was introduced as a measure to control the amount of faecal contamination on hides (Leitch et al. (2001)). However, feed deprivation is thought to predispose cattle to carriage of E. coli and Salmonella (Brownlie and Grau (1967)).

There is thus a continuing need for new and improved compositions capable of reducing the numbers of, or inactivating, bacterial pathogens (including those present at any point in the food chain, or present in the environment). This includes the processes involved in the preparation of meat and vegetable products for the consumer, and the control of bacterial pathogens present within animals which carry and shed micro-organisms (including, for example, humans, cattle, pigs, sheep, chickens, and turkeys). It also includes the prevention of proliferation or the reduction in survival of pathogens in animal feeds and the decontamination of water troughs through which infectious agents may spread from animal to animal or from animal to human. It especially includes applications to reduce the presence of pathogenic bacteria on the surfaces of fruits and vegetables and on surfaces (for example floors, benches, work tops, walls, cutting implements), or present on the surface of fish, shellfish, raw cut meat products and animal carcasses.

With regard to reducing or eliminating bacterial pathogens in the preparation of meat and vegetable products for the consumer, the various potential control stages in the food chain are referred to as Hazard Analysis Critical Control Points (HACCP). E. coli O157 is an important pathogen and is thus of particular interest, but other bacterial pathogens which may be carried and shed from host animals include, for example, Salmonella species (including S. enteritidis), Campylobacter spp., Staphylococcus spp., Listeria monocytogenes as well as non-O157 E. coli strains causing food-borne infections.

The technology developed to control food borne pathogens could also find application for other bacterial pathogens. Compounds or proceses effective against the range of bacterial pathogens mentioned above would be likely to be active against other bacterial pathogens found in habitats other than food. An example is the control of methicillin-resistant Staphylococcus aureus (MRSA). This bacterium can be found on unprotected surfaces in hospitals (floors and walls, implements etc) and novel disinfectants could be invaluable in its control.

Currently there are limited and relatively inefficient means of controlling the contamination of the food chain and the environment by E. coli O157 and other pathogenic bacteria, treating human infection, and reducing carriage of pathogenic bacteria by animals such as cattle, sheep, and humans. Indeed, certain antibiotic therapies for treatment of humans infected with E. coli O157 causes lysis of the bacterium and subsequent release of the potent disease-causing toxin.

Current methods of reducing and killing bacterial pathogens on surfaces such as abattoir and butchers' floors and work benches comprise washing and soaking such surfaces in chlorinated solutions. However, reports have suggested that chlorine does not effectively kill E. coli O157. Furthermore, resistance to this chemical may occur (see Beuchat (1999) Journal of Food Protection 62(8): 845-849; Cutter et al. (1995) Journal of Food Safety 15(1): 67-75; Lisle et al. (1998) Applied and Environmental Microbiology 64(12): 4658-4662; and Zhao et al. (2001) Journal of Food Protection 64(10): 1607-1609). In addition, chlorine can alter the taste and smell of foods in contact with the surface, reducing palatability. These disadvantages are also inherent in the use of chlorinated water for the washing of fruits, vegetable leaves and similar materials.

There are a number of publications describing the inhibition of bacterial pathogens on fresh food items. WO-A-99/44444 describes the use of a solution containing lactic acid and at least one ingredient chosen from hydrogen peroxide, sodium benzoate or glycerolmonolaurate at certain temperatures and durations. No mention is made of the distinction between the L- and D-isomers of lactic acid, nor is there any mention of coumarin compounds.

Castillo et al. (2001) describe the use of L-lactate in reducing the presence of pathogenic bacteria on hot beef carcasses and suggest that the technology may be useful at lower temperatures. No mention is made as to an additive effect of L-lactate with coumarin compounds, nor is there any mention as to the use of L-lactate in reducing, inhibiting or killing bacteria in vivo in animals or humans. Certain companies and researchers have published the use of Lactobacillus species (Nutraceutix Inc.) and certain E. coli species (U.S. Pat. No. 5,965,128) in inhibiting E. coli O157 in vitro and in vivo. Duncan (1998) describes the inhibitory effect of esculetin, a coumarin compound, in combination with the volatile fatty acids (VFA's) acetate, butyrate and propionate towards E. coli O157. There is no mention of other organic acids, such as lactic acid, having any additive or synergistic effect on the inhibitory and killing of E. coli O157. In addition, the most active VFA is butyrate which has an associated undesirable smell rendering this acid unsuitable for use in the food industry at the concentrations required.

Bintsis et al. (2000) show the efficacy of furocoumarins in inhibiting certain pathogens, including E. coli O157. However, there is no mention of the combination of coumarins with lactic acid or other organic acids.

We have now found that the L-isomer of lactate is unexpectedly more active than D-lactate against pathogenic E. coli strains (including E. coli O157). Further, we have found that combination of an organic acid with a coumarin or a glycoside thereof results in a synergistically enhanced anti-bacterial effect. The “organic acid” as herein defined may be any organic acid but specifically excludes the volatile fatty acids acetate, propionate and butyrate. In one embodiment the organic acid may be any organic acid but excludes short chain volatile fatty acids. Desirably, the organic acids selected are medium chain fatty acids (eg. heptanoic and decanoic acids), long chain fatty acids (eg. dodecanoic acid), unsaturated acids (eg. sorbic acid), hydroxylic acids (eg. lactic acid and citric acid), aromatic acids (eg. benzoic acid and salicyclic acid) or multicarboxylic acids (eg. citric acid and succinic acid). Preferably the organic acids are hydroxylic acids such as citrate or lactate. L-isomers of optically active hydroxylic acids are preferred. It should be noted that the nomenclature for fatty acids adopted is as set out by Cherrington et al. (1991).

The present invention provides an anti-bacterial composition comprising an admixture of an organic acid as defined above and a coumarin or a coumarin glycoside. Lactate, citrate and benzoic acid are preferred, especially L-lactate.

The present invention also provides a method for reducing the infective ability or inactivating bacterial pathogens by contacting said bacteria with a mixture of an organic acid as defined above and a coumarin or a coumarin glycoside. Further, the method and composition of the present invention may be used to reduce the shedding of pathogens (including but not limited to E. coli O157, Salmonella, Listeria, Campylobacter and MRSA) from animals and humans.

We believe that any coumarin will be effective in the composition described above. The preferred coumarins are esculetin, scopoletin, umbelliferone and Coumarin, but other coumarins may also be used. The coumarins are derivatives of benzo-α-pyrone and occur in plants in the free state and as glycosides. For clarity, the term “coumarin” will be used to describe the generic group of benzo-α-pyrone compounds, whereas “Coumarin” will refer to 1,2 benzopyrone. In addition to the free state coumarins, the coumarin glycosides may also be used since these are commonly converted to the free coumarin in vivo and may have the advantage of increased solubility which aids administration and absorbance. Additionally, the sugar moiety, once hydrolysed from the glycoside, can provide a sugar source for growth of beneficial bacteria. An example of a coumarin glycoside is esculin, which is a glycoside of esculetin. Furocoumarins, for example psoralen, 3-methoxy-psolaren and 8-methoxy-psoralen may also be of interest.

Preferably, the composition contains from 1 mM to 500 mM, more preferably from 20 mM or 50 mM to 250 mM, of organic acid. Preferred organic acids are lactate, citrate and benzoic acid.

Preferably, the composition contains from 0.05 mM to 15 mM of a coumarin or a coumarin glycoside, preferably at least 0.5 mM, for example 0.68 mM, of a coumarin or a coumarin glycoside.

The composition of the present invention has the advantage of being applicable to all stages of potential contamination and cross-contamination from farm to fork. Exemplary stages include use in the treatment of salad leaves and other vegetable material, in animal feeds, in water troughs on farms, in food preparation from slaughter to sale to the public, for use with plant matter intended as a foodstuff and also for treatment of animals or humans infected with said pathogens. Further, the composition could be used as a disinfectant, cleaner, sterilizer for commercial and non-commercial cooling devices such as fridges and freezers. In particular, the composition could be used to disinfect buildings, in particular public buildings such as schools or hospitals, or to disinfect surfaces (such as floor, walls, furniture and medical devices/implements). The composition could further be used in packaging material for foods (such as plastic “clingfilm” wrap) and, since the composition increases in effectiveness with increasing temperature, would protect wrapped produce taken from the refrigerated conditions in retail premises during transport. The composition could also find use in washing, coating or being incorporated into bandages, dressings and other coverings used to protect wounds from infection and contamination.

One of the benefits of the composition of the present invention resides with the antioxidant properties of coumarins. Antioxidants have well known health enhancing and disease preventing properties by virtue in part to their ability to reduce oxidative damage to our cells which may lead to development of many clinical conditions including cancers, heart disease, Alzheimer's disease and arthritis. Accordingly, in addition to the ability of coumarins to inhibit and kill pathogens, ingestion by animals and humans will enhance the health status of the recipient and may help to prevent and treat diseases related to oxidative stress. Apart from the possible benefits to health outlined above, the antioxidant properties of the coumarins are likely to extend the shelf-life of treated food or feed stuffs.

The composition of the present invention may be administered to animals to reduce shedding of the pathogens into the environment, for example fields, farms, water supplies, slurry and vehicles used for transportation of animals. Reduction of shedding leads to a lowering of the risk of contamination of the environment and edible and non-edible agricultural products such as fruit and vegetables and will also reduce the risk of infection to other animals and humans.

Preferably the method of the present invention is conducted at a temperature of 37° C. or lower, for example 20° C. or 5° C. (representing body temperature, ambient temperature and refrigerated temperature respectively). In general, increasing the temperature increases the effectiveness of the composition. However, even at 5° C., the numbers of Salmonella, for example, are reduced by greater than 5 log₁₀ units within 60 minutes. At 20° C., a greater than 5 log₁₀ unit decrease occurs in 5 minutes.

Further the present invention may be administered to humans infected with pathogens so as to reduce the numbers, inhibit and kill the pathogens and consequently treat diseases caused by the infecting organism. For example the composition of the invention could be used to treat humans infected with E. coli O157 or MRSA and thus prevent or ameliorate the effects of infection with such pathogens, which include haemorrhagic colitis and haemolytic uremic syndrome and sepicaemia.

In one embodiment, the composition of the present invention may further comprise a volatile fatty acid (VFA) such as, for example, acetate, butyrate or propionate. Alternatively or additionally the composition may comprise a polyasaccharide or other readily fermentable compound which, upon digestion in the gut, is converted to an acid such as lactate.

The composition can be used in various formats for example as sprays, liquid solutions, gels, packaging and wrapping material for foods for use on surfaces and can be delivered to the site of action in the rumen or gastro-intestinal tract by oral administration in any appropriate carrier, excipient, diluent or stabilizer. Such delivery mechanisms may be of any formulation including but not limited to solid formulations such as tablets or capsules or as feed additives; liquid solutions such as yoghurt or drinks or suspensions.

It is believed that any bacterial pathogen will be adversely affected by the composition of the present invention. Pathogenic E. coli strains, including E. coli O157, are of particular interest, as are Salmonella spp, Listeria spp. and Staphylococcus spp., especially MRSA.

The present invention will now be further described with reference to the following, non-limiting, examples and figures in which:

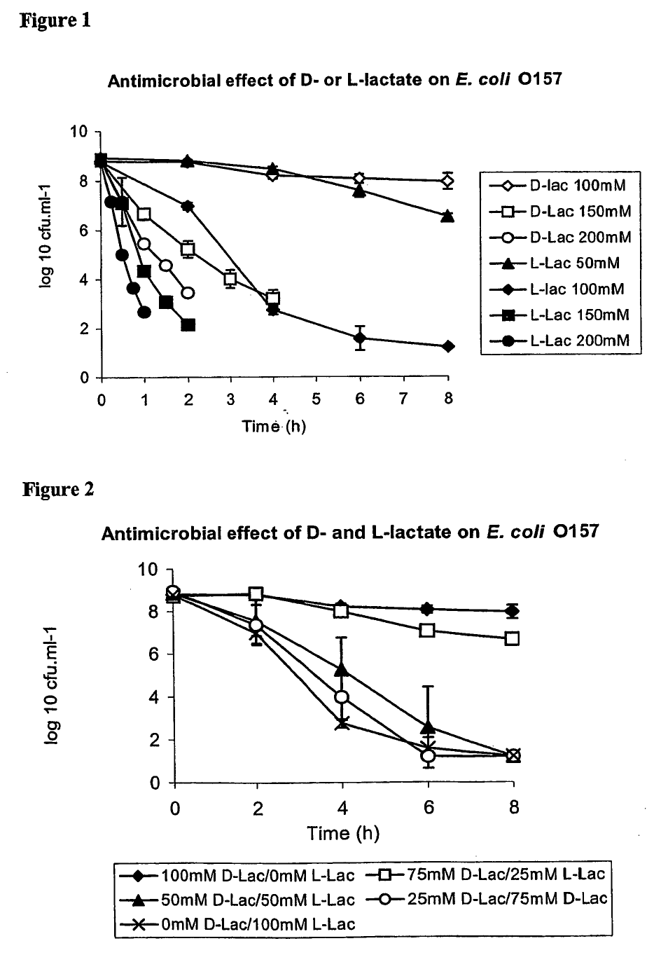

FIG. 1 shows the susceptibility of E. coli O157:H7 strain NCTC 12900 to L-lactate and D-lactate. The L-lactate solution concentrations were 50, 100, 150 and 200 mM and those of D-lactate 100, 150 and 200 mM. The solutions all had a final pH of 3.8 and were incubated at 37° C. The limit of detection is 50 cfu/ml or 1.7 log₁₀ cfu/ml.

FIG. 2 illustrates the susceptibility of E. coli O157:H7 strain NCTC 12900 to various proportions of L-lactate and D-lactate. The solution concentrations were 100 mM D-lactate, 75 mM D-lactate+25 mM L-lactate, 50 mM D-lactate+50 mM L-lactate, 25 mM D-lactate+75 mM L-lactate, and 100 mM L-lactate. The solutions all had a final pH of 3.8 and were incubated at 37° C. The limit of detection is 50 cfu/ml or 1.7 log₁₀ cfu/ml.

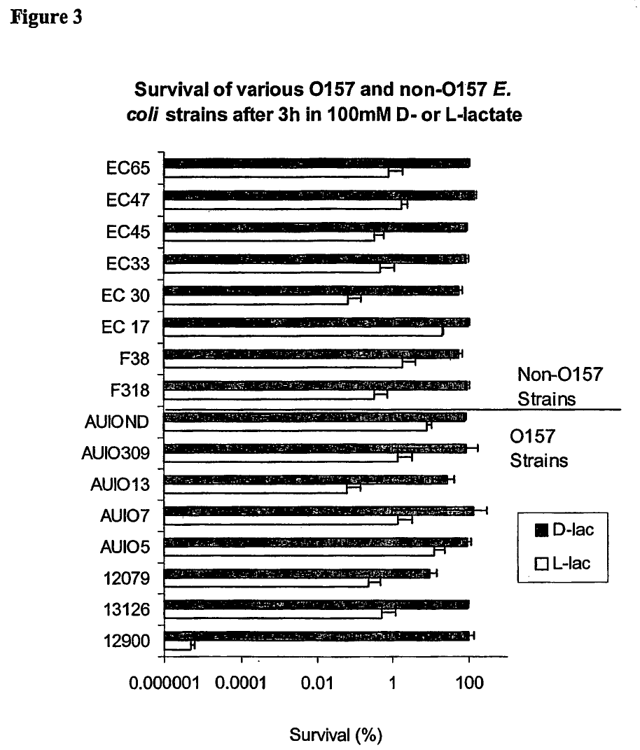

FIG. 3 indicates the survival of 8 E. coli O157:H7 strains and 8 non-O157 E. coli strains following treatment with 100 mM L-lactate or D-lactate for 3 hours. The solutions all had a final pH of 3.8 and were incubated at 37° C. The limit of detection is 50 cfu/ml or 1.7 log₁₀ cfu/ml.
Dark Shading=D-Lactate.
Light Shading=L-Lactate.

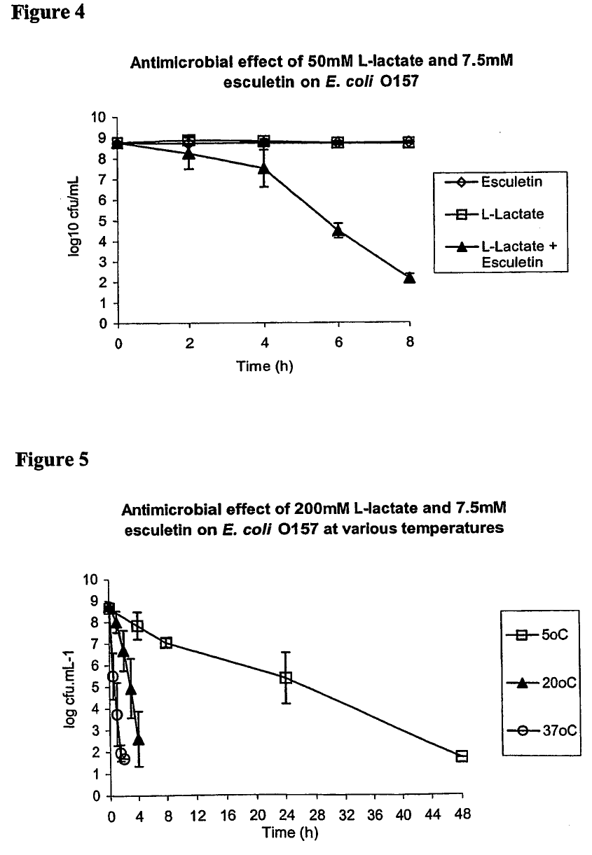

FIG. 4 illustrates the synergy between L-lactate (50 mM) and esculetin (7.5 mM) in reducing the numbers of the E. coli O157:H7 strain NCTC 12900. The solutions all had a final pH of 3.8, were incubated at 37° C. and the limit of detection was 50 cfu/ml or 1.7 log₁₀ cfu/ml.

FIG. 5 illustrates the effect of temperature on the antimicrobial efficacy of 200 mM L-lactate and 7.5 mM esculetin on the E. coli 0157:H7 strain NCTC 12900. Cultures were incubated at 5, 20 and 37° C. The solutions all had a final pH of 3.8 and the limit of detection was 50 cfu/ml or 1.7 log₁₀ cfu/ml.

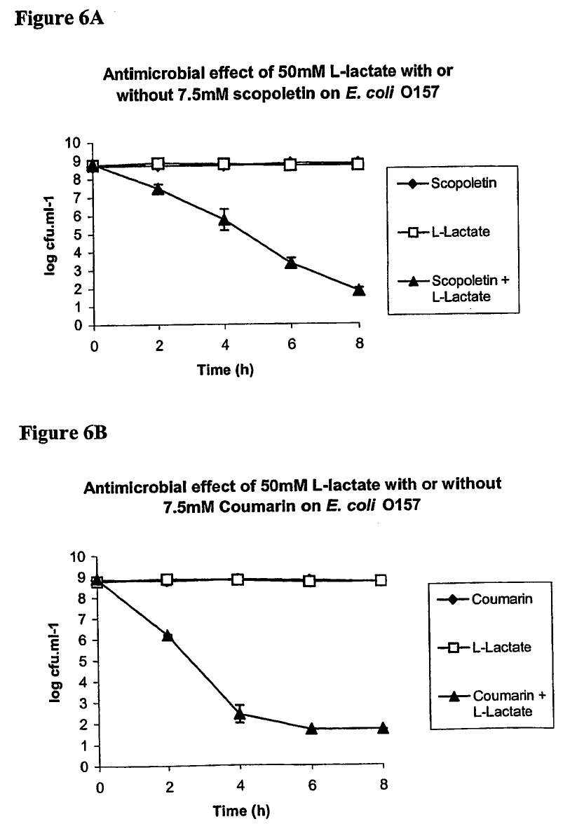

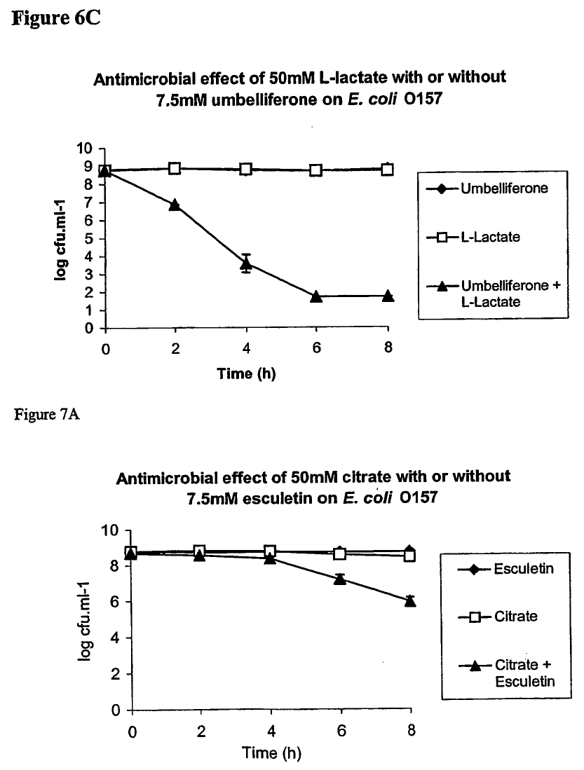

FIGS. 6A-C depict the synergism between various coumarins (7.5 mM) and L-lactate (50 mM) against the E. coli 0157:H7 strain NCTC 12900. The coumarins tested were scopoletin (A), Coumarin (B) and umbelliferone (C). Cultures were incubated at 37° C., had a final pH of 3.8 and the limit of detection was 50 cfu/ml or 1.7 log₁₀ cfu/ml.

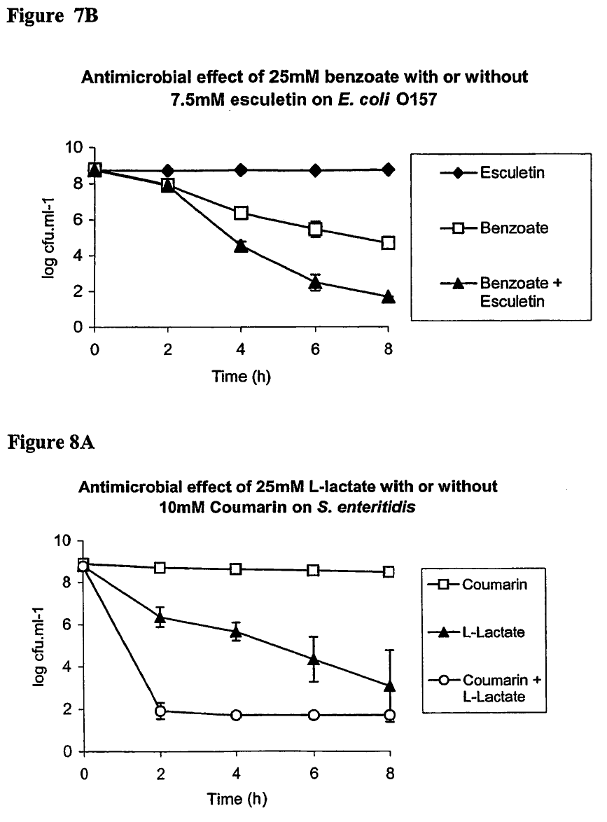

FIGS. 7A-B illustrate the synergy between esculetin and 50 mM citric acid (A) or 25 mM benzoic acid in reducing the numbers of the E. coli 0157:H7 strain NCTC 12900. The solutions all contained 7.5 mM esculetin and had a final pH of 3.8. The limit of detection was 50 cfu/ml or 1.7 log₁₀ cfu/ml.

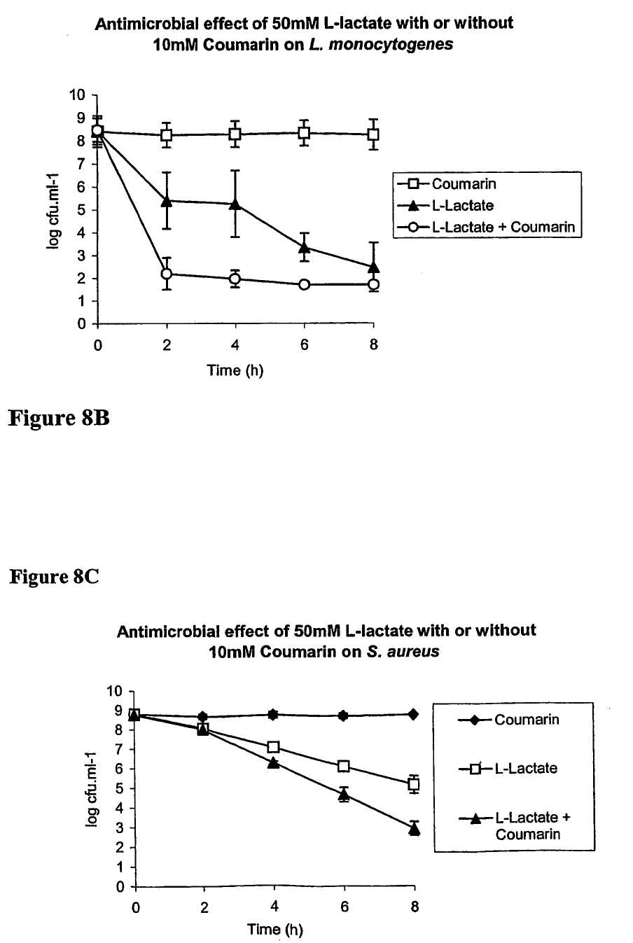

FIGS. 8A-C indicates the synergy between Coumarin and L-lactate for S. enteritidis (A), L-monocytogenes (B), and an MRSA strain of S. aureus (C). The solutions all contained 10 mM Coumarin and had a final pH of 3.8. The limit of detection was 50 cfu/ml or 1.7 log₁₀ cfu/ml.

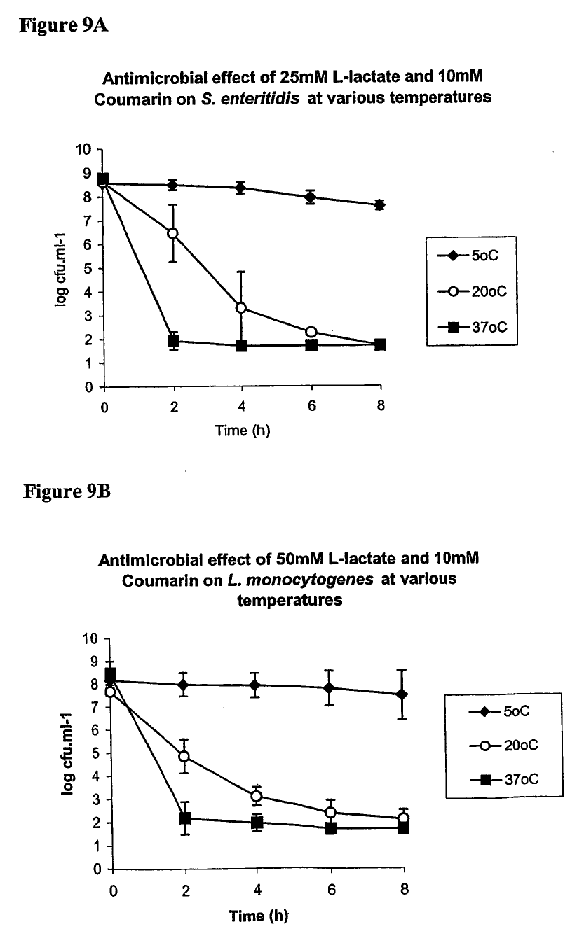

FIGS. 9A-B compare the effect of temperature on survival of S. enteritidis (A) and L-monocytogenes (B) in L-lactate and Coumarin. The solutions contained 10 mM Coumarin and 25 mM L-lactate (A) or 50 mM L-lactate (B). The final pH was 3.8 and the limit of detection 50 cfu/ml or 1.7 log₁₀ cfu/ml.

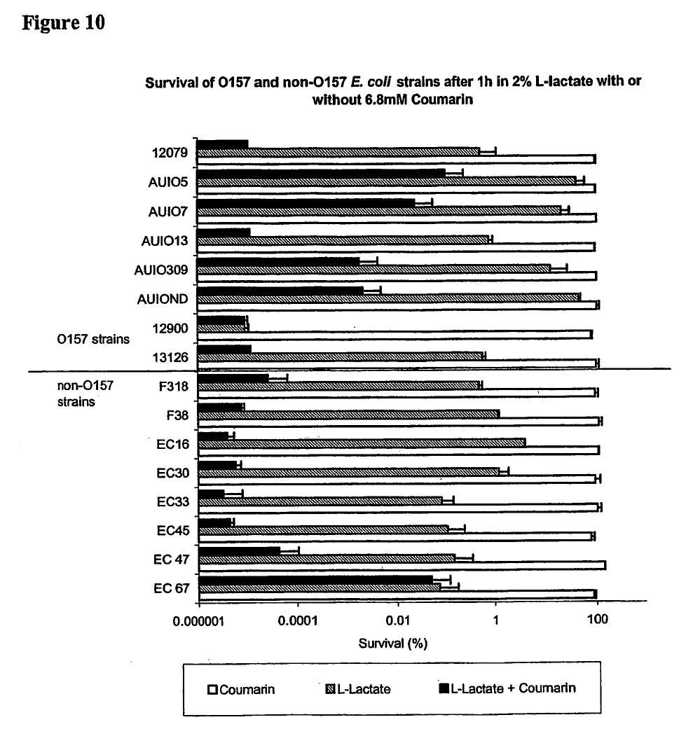

FIG. 10 illustrates the synergy between 2% L-lactate and 6.8 mM Coumarin in reducing the viability of 8 E. coli O157:H7 strains and 8 non-O157:H7 E. coli strains determined over 1 hour. Results are shown as percentage survival.

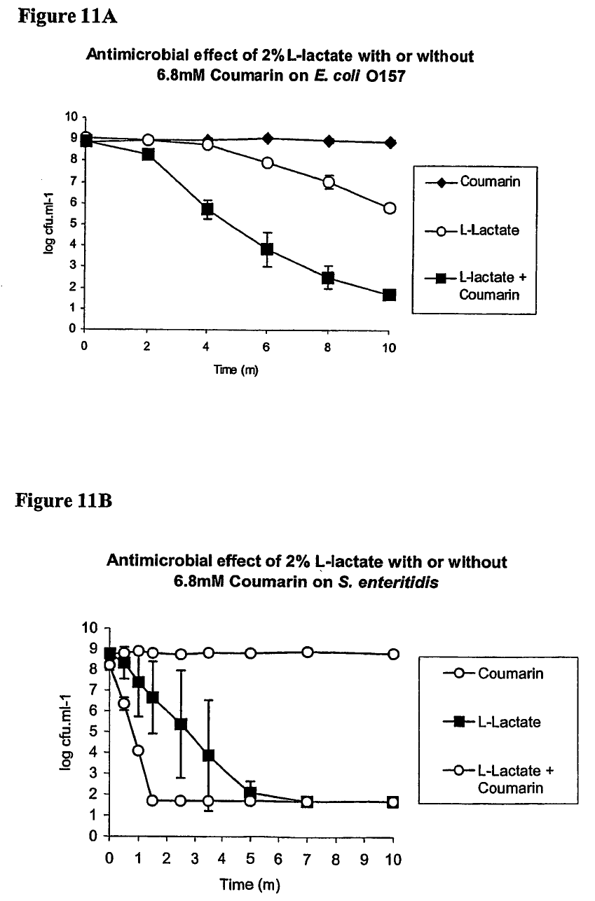

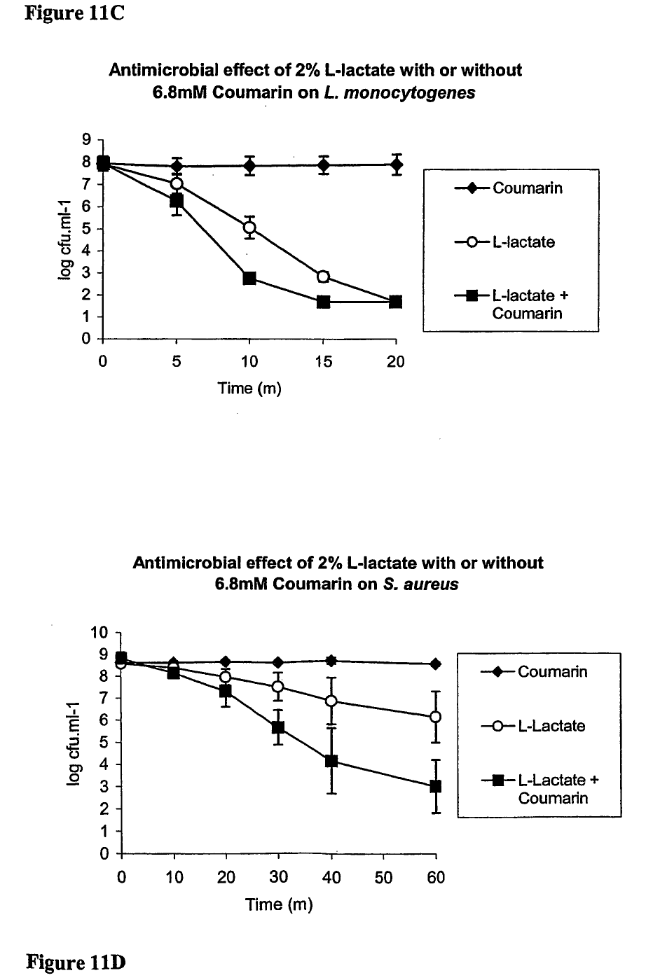

FIGS. 11A-D show the synergistic antimicrobial effect of 2% L-lactate and 6.8 mM Coumarin on the E. coli 0157 strain NCTC 12900 (A), S. enteritidis (B), L-monocytogenes (C) and S. aureus (D). The limit of detection was 50 cfu/ml or 1.7 log₁₀ cfu/ml.

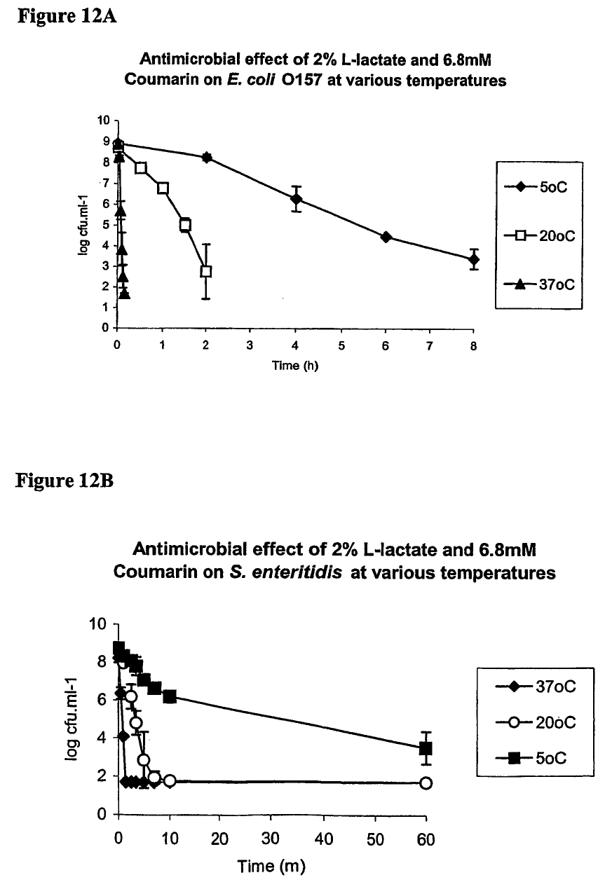

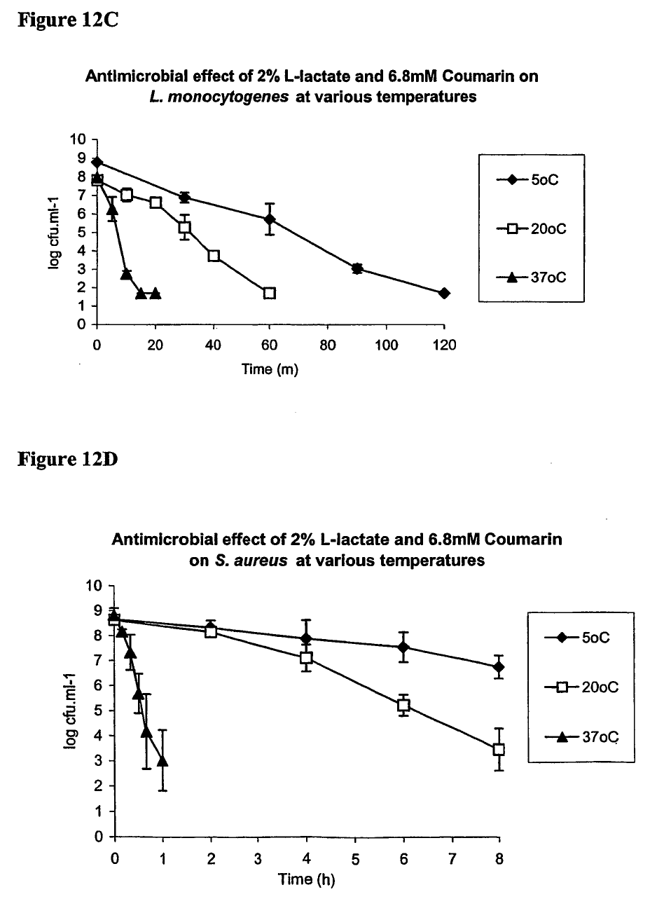

FIGS. 12A-D illustrate the effect of temperature on the antimicrobial efficacy of 2% L-lactate and 6.8 mM Coumarin for the E. coli 0157:H7 strain NCTC 12900 (A), S. enteritidis (B), L. monocytogenes (C) and S. aureus (D). The limit of detection was 50 cfu/ml or 1.7 log₁₀ cfu/ml.

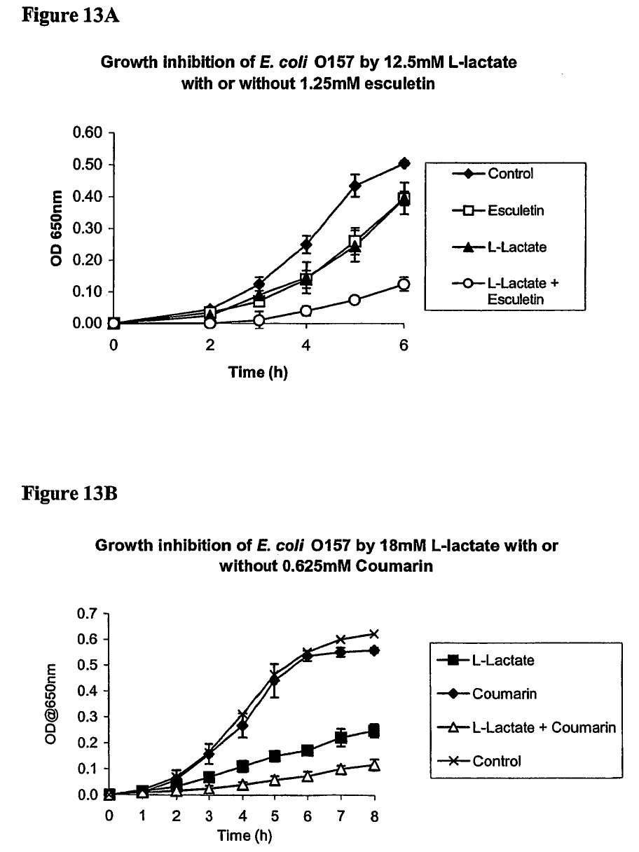

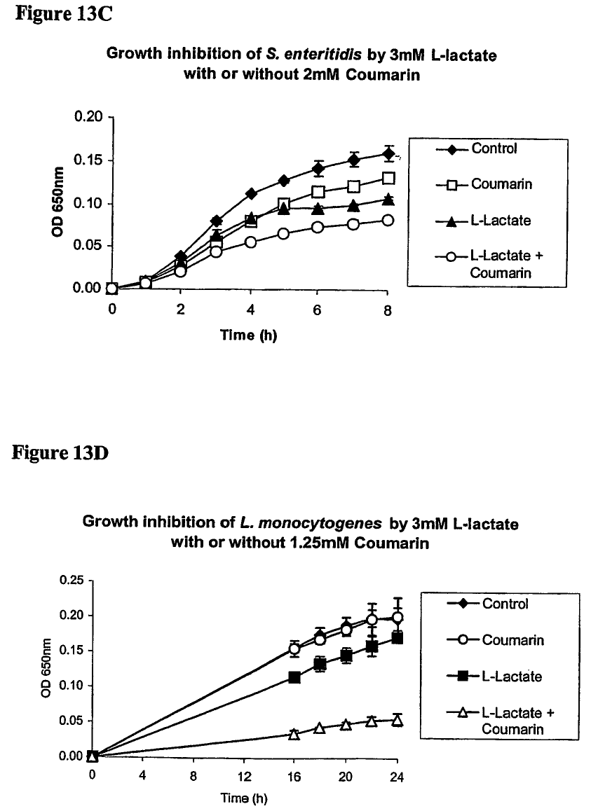

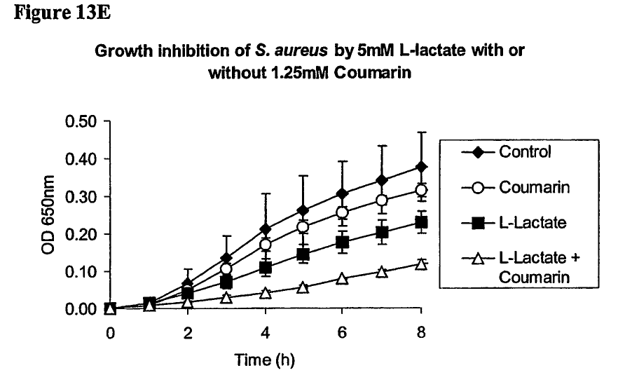

FIGS. 13A-E demonstrate the synergistic growth inhibition of L-lactate and esculetin (A) or L-lactate and Coumarin (B-E) on E. coli O157:H7 strain NCTC 12900 (A-B), S. enteritidis (C), L. monocytogenes (D) and S. aureus (E).

### EXAMPLES

Table 1 shows the bacterial strains used in the examples.

TABLE 1

| E. coli Strains | | | Other Strains | |
|---|---|---|---|---|
| Strain | Origin | Serotype | Species | Strain |
| NCTC | Human | O157:H7 | Salmonella | NCTC |
| 12900 | | | enteritidis | 4444 |
| NCTC | Human | O157:H7 | Listeria | NCTC |
| 13126 | | | monocytogenes | 11994 |
| NCTC | Human | O157:H7 | Staphylococcus | NCTC |
| 12079 | | | aureus | 10442 |
| AUIO-5 | Cattle | O157:H7 | | |
| | faeces | | | |
| AUIO-7 | Raw milk | O157:H7 | | |
| AUIO-13 | Minced | O157:H7 | | |
| | beef | | | |
| AUIO-309 | Cheese | O157:H7 | | |
| AUIO-ND | Sheep | O157:H7 | | |
| | faeces | | | |
| F318 | Sheep | O162 | | |
| | rumen | | | |
| F38 | Sheep | O rough | | |
| | rumen | | | |
| EC17 | Pig | O106:NM | | |
| EC30 | Bison | O113:H21 | | |
| EC33 | Sheep | O7:H21 | | |
| EC45 | Pig | ON:HM | | |
| EC47 | Sheep | ON:H18 | | |
| EC67 | Goat | O4:H43 | | |

Methods and Results

As the infectious dose of E. coli O157:H7 is very low, our main aim was to develop a treatment capable of killing high levels of E. coli cells. The E. coli strains were cultured such that there was a population of approximately 10⁹ cfu/ml. The final pH for each culture was 3.8, except where 2% lactate was present in which case the final pH was around 2.0. Treatments consisted of various concentrations of the organic acids L-lactate, D-lactate, citrate or benzoate and the coumarins esculetin, Coumarin, scopoletin or umbelliferone. These were added to cultures which were then incubated at 5° C., 20° C. or 37° C. Samples were extracted at various time intervals and the population of E. coli O157:H7 was determined. Other pathogens were tested in a similar manner with the exception of L. monocytogenes where the starting population was 10⁸ cfu/ml.

L-lactate (final concentrations of 50, 100, 150 or 200 mM) or D-lactate (final concentrations of 100, 150 or 200 mM) were added to prepared cultures and incubated at 37° C. As illustrated in FIG. 1, L-lactate was more effective at reducing numbers of the E. coli O157:H7 strain NCTC 12900 than D-lactate at all concentrations measured. The inactivation of this organism by either treatment was dose-dependent.

L-lactate and D-lactate (final concentrations of 100 mM D-lactate, 75 mM D-lactate+25 mM L-lactate, 50 mM D-lactate+50 mM L-lactate, 25 mM D-lactate+75 mM L-lactate, and 100 mM L-lactate) were added to prepared cultures and incubated at 37° C. As shown in FIG. 2, 100 mM L-lactate exerted a greater antimicrobial effect than 100 mM D-lactate on E. coli 0157:H7 strain NCTC 12900. Increasing the proportion of the L-isomer over the D-isomer increased the antimicrobial efficacy in a dose-dependent manner for both strains.

Treatments consisting of 100 mM of L-lactate or D-lactate were tested against 8 E. coli O157:H7 strains and 8 non-O157:H7 E. coli strains. The viability was determined initially and after 3 hours at 37° C. The percentage survival was calculated by dividing the final viability by the initial viability and multiplying the result by one hundred. As shown in FIG. 3, the inactivation caused by L-lactate was much greater than that of D-lactate for all the E. coli strains tested. This strongly suggests that the greater susceptibility to L-lactate compared to D-lactate is widespread in E. coli.

The effect of L-lactate (50 mM) and esculetin (7.5 mM) on strain NCTC 12900 at 37° C. was determined and the results are shown in FIG. 4. In combination, these compounds synergistically reduced the survival of strain NCTC 12900 by approximately 7 log₁₀ units/ml in 8 hours. The combined treatment of L-lactate lactate and esculetin had a greater effect than the individual treatments, illustrating synergy between the two compounds.

The effect of temperature on the antimicrobial efficacy of 200 mM L-lactate+7.5 mM esculetin on the E. coli O157:H7 strain NCTC 12900 was tested. The temperatures assayed were 5° C., 20° C. and 37° C. As shown in FIG. 5, as the temperature increased, the efficacy of the antimicrobial compounds increased.

The range of coumarins tested was extended to include scopoletin, Coumarin and umbelliferone and their potential synergy with L-lactate against E. coli O157:H7 strain NCTC 12900 was evaluated. As shown in FIG. 6A, neither L-lactate (50 mM) nor scopoletin (7.5 mM) on their own affected the viability of this bacterial strain. In combination these compounds reduced the numbers of NCTC 12900 by greater than 7 log₁₀ units in 8 hours Similarly, neither 7.5 mM Coumarin (FIG. 6B) nor 7.5 mM umbelliferone (FIG. 6C) affected viability, but in concert with L-lactate, these compounds caused a synergistic reduction in viable E. coli O157 cells of greater than 7 log₁₀ units in 6 hours. This strongly suggests that coumarins in general exhibit an antimicrobial synergy with L-lactate for E. coli strains.

The range of organic acids tested was extended to include citrate and benzoate and their potential synergy with 7.5 mM esculetin against NCTC 12900 evaluated. As shown in FIG. 7A, neither citrate (50 mM) nor esculetin (7.5 mM) when used individually affected the viability of the E. coli O157:H7 strain. In combination these compounds reduced the numbers of NCTC 12900 by approximately 3 log₁₀ units in 8 hours. As shown in FIG. 7B, benzoate (25 mM) on its own reduced viability by approximately 4 log₁₀ units in 8 hours. Benzoate with esculetin (7.5 mM) caused a synergistic reduction in viable E. coli O157 cells of greater than 7 log₁₀ units in 8 hours. This strongly suggests that organic acids in general exhibit an antimicrobial synergy with coumarins for E. coli strains.

The antimicrobial effect of L-lactate and Coumarin on pathogens other than E. coli was examined. The strains examined were Salmonella enteritidis NCTC 4444, Listeria monocytogenes NCTC 11994 and the methicillin-resistant Staphylococcus aureus (MRSA) strain NCTC 10442.

As shown in FIG. 8A, Coumarin (10 mM) alone did not affect the viability of S. enteritidis whereas L-lactate (25 mM) reduced viability by greater than 5 log₁₀ units in 8 hours. Together, Coumarin and L-lactate synergistically reduced the viability of this organism by greater than 7 log₁₀ units in 2 hours.

FIG. 8B illustrates the affect of Coumarin and L-lactate on L. monocytogenes. Coumarin (10 mM) alone had no effect on viability whereas L-lactate (50 mM) reduced viability by greater than 5 log₁₀ units in 8 hours. Coumarin and L-lactate together synergistically reduced the viability of this L. monocytogenes strain by greater than 6 log₁₀ units in 2 hours.

The viability of S. aureus (FIG. 8C) was not affected by 10 mM Coumarin alone whereas 50 mM L-lactate reduced viability by greater than 3 log₁₀ units in 8 hours. The viability of this MRSA strain was reduced by approximately 6 log₁₀ units in 8 hours when both L-lactate and Coumarin were present. These results strongly suggest that organic acids and coumarins exhibit an antimicrobial synergy for bacterial pathogens in general.

The effect of temperature on the antimicrobial efficacy of L-lactate and Coumarin was examined for the S. enteritidis strain (FIG. 9A) and the L. monocytogenes strain (FIG. 9B). For both of these organisms the antimicrobial effect was greater as the temperature increased.

For certain applications it was desirable to determine potential synergy between L-lactate and Coumarin at the concentrations of these compounds likely to be used in commercial-like environments. The concentration of L-lactate was 2% and that of Coumarin 6.8 mM. As shown in FIG. 10, a range of 8 E. coli O157 strains and 8 non-O157 E. coli strains were tested against these compounds and the viability of the strains determined at the start of the experiment and after 1 hour. Survival was calculated as previously. Coumarin alone had little effect on the survival of the E. coli strains whereas L-lactate reduced survival to between 0.00001% and 46%. L-lactate and Coumarin in combination reduced survival to between 0.00001% and 0.1%. For all strains tested, the antimicrobial effect of L-lactate combined with Coumarin was greater than either of the compounds tested individually. This strongly suggests that L-lactate and Coumarin at commercially-applicable concentrations exert a synergistic antimicrobial effect on E. coli strains.

The viability of various bacterial species in commercially applicable concentrations of L-lactate and Coumarin was examined. Coumarin (6.8 mM) had no effect on the viability of the E. coli O157:H7 strain NCTC 12900 (FIG. 11A) whereas L-lactate (2%) caused a decrease in viability of approximately 3 log₁₀ units in 10 minutes. Together, L-lactate and Coumarin decreased the viability of this E. coli O157:H7 strain by greater than 7 log₁₀ units in 10 minutes. As shown in FIG. 11B, Coumarin (6.8 mM) alone had no effect on the viability of S. enteritidis whereas L-lactate reduced bacterial numbers by greater than 7 log₁₀ units in 5 minutes. The combination of Coumarin and L-lactate reduced viability by greater than 7 log₁₀ units in 1.5 minutes. As shown in FIG. 11C, Coumarin (6.8 mM) had no effect on the viability of L. monocytogenes whereas L-lactate on its own reduced viability by greater than 7 log₁₀ units in 20 minutes. Together Coumarin and L-lactate reduced the viability of this pathogen by greater than 7 log₁₀ units in 15 minutes. Coumarin had no effect on the viability of S. aureus (FIG. 11D) whereas L-lactate alone reduced viability by approximately 2 log₁₀ units in 60 minutes. In combination, Coumarin and L-lactate reduced viability of this organism by greater than 5 log₁₀ units in 60 minutes. This strongly suggests that organic acids and Coumarin at commercially-applicable concentrations exert a synergistic antimicrobial effect on all bacterial pathogens.

The effect of temperature on the efficacy of commercially-applicable concentrations of L-lactate (2%) and Coumarin (6.8 mM) on various pathogens was investigated. As shown in FIG. 12A, the viability of the E. coli O157 strain NCTC 12900 at 37° C. was reduced by greater than 7 log₁₀ units in 10 minutes. At 20° C., a greater than 5 log₁₀ unit reduction in viability was achieved in 2 hours and at 5° C. a greater than 5 log₁₀ unit reduction was achieved in 8 hours. The viability of S. enteritidis (FIG. 12B) was reduced by greater than 7 log₁₀ units in 1.5 minutes at 37° C. and by a similar extent in 7.5 minutes at 20° C. At 5° C., viability was reduced by approximately 5 log₁₀ units in 60 minutes. The viability of L. monocytogenes (FIG. 12C) was reduced by greater than 7 log₁₀ units in 15 minutes at 37° C. The same extent of reduction was achieved after 60 minutes at 20° C. and after 120 minutes at 5° C. As shown in FIG. 12D, Coumarin and L-lactate reduced the viability of S. aureus by greater than 5 log₁₀ units in 1 hour at 37° C. At 20° C., viability was reduced by 5 log₁₀ units in 8 hours whereas at 5° C. viability was reduced by approximately 2 log₁₀ units in 8 hours.

For certain applications it is desirable to determine the potential for contaminating E. coli O157:H7 strains to increase in numbers. To evaluate the effect of L-lactate and coumarins on growing E. coli O157:H7 cells, NCTC 12900 was prepared as for the above experiments. The culture was then diluted into fresh media and incubated at 37° C. for 2 hours. L-lactate and/or a coumarin were added and the cultures were re-incubated over a period of time. Bacterial growth was monitored by spectrophotometer (650 nm) and compared to a control lacking both L-lactate and a coumarin.

As illustrated in FIG. 13A, 12.5 mM L-lactate or 1.25 mM esculetin individually caused a small reduction in the growth of the E. coli O157:H7 strain NCTC 12900 compared to the control. L-Lactate and esculetin added to the culture together had a synergistic effect, almost entirely retarding the growth of this strain. Compared to the control, 0.625 mM of Coumarin only slightly reduced the extent of growth of NCTC 12900 (FIG. 13B). Growth of this strain was inhibited substantially by 18 mM L-lactate and the combination of Coumarin and L-lactate caused an even greater reduction in growth. As shown in FIG. 13C, Coumarin (2 mM) inhibited the growth of S. enteritidis slightly and L-lactate inhibited growth to a greater extent. Together, these compounds reduced the growth of S. enteritidis more than either compound on its own. As shown in FIG. 13D, 1.25 mM Coumarin did not affect the growth of L. monocytogenes and L-lactate inhibited growth slightly. Together Coumarin and L-lactate reduced the growth of this organism substantially. The growth of S. aureus (FIG. 13E) was inhibited by Coumarin (1.25 mM) on its own and by L-lactate (5 mM) on its own. Together these compounds caused a synergistic decrease in the growth of this species. These results strongly suggest that organic acids and coumarins synergistically inhibit growth of all bacterial pathogens.

### REFERENCES

Bintsis et al. (2000) Food Microbiol. 17, 687-695.

Brownlie, L. E. and Grau, F. H. (1967) Effect of food intake on growth and survival of salmonellas and Escherichia coli in the bovine rumen. J. Gen. Microbiol. 46, 125-134.

Castillo A. et al. (2001). Lactic acid sprays reduce bacterial pathogens on cold beef carcass surfaces and in subsequently produced ground beef. J. Food Prot. 64, 58-62.

Cherrington C. A. et al. (1991). Organic acids: chemistry, antibacterial activity and practical applications. Advances in Microbial Physiology 32: 87-108.

Duncan Sylvia H., Flint, Harry J., and Stewart, Colin. S. (1998) Inhibitory activity of gut bacteria against Escherichia coli O157 mediated by dietary plant metabolites. FEMS Microbiol. Ltt. 164, 283-288.

LeJeune, J. T., Besser, T. E. and Hancock, D. D. (2001) Cattle water troughs as reservoirs of Escherichia coli O157. Appl. Environ. Microbiol. 67, 3053-3057.

Leitch, E. C. McW., Duncan, S. H., Stanley, K. N. and Stewart, C. S. (2001) Dietary effects on the microbiological safety of food. Proceed. Nutrit. Soc. 60, 247-255.

1. An anti-bacterial composition comprising an admixture of an organic acid and a coumarin or coumarin glycoside, wherein the organic acid is other than a short chain fatty acid.

2. The composition as claimed in claim 1 wherein the organic acid is heptanoic acid, decanoic acid, dodescanoic acid, sorbic acid, lactic acid, citric acid, benzoic acid, salicylic acid or succinic acid.

3. The composition as claimed in claim 2 wherein the organic acid is lactate, citrate or benzoic acid.

4. The composition as claimed claim 1 comprising 1 to 500 mM of the organic acid.

5. The composition as claimed in claim 4 comprising 20 mM to 250 mM of the organic acid.

6. The composition as claimed in claim 5 comprising 20 mM to 250 mM of L-lactate.

7. The composition as claimed claim 1 wherein the coumarin or coumarin glycoside is esculetin, scopoletin, umbelliferone, Coumarin, esculin or mixtures thereof.

8. The composition as claimed in claim 7 wherein the coumarin or coumarin glycoside is Coumarin.

9. The composition as claimed in claim 1 comprising 0.05 mM to 15 mM of a coumarin or coumarin glycoside.

10. The composition as claimed in claim 1 comprising at least 0.5 mM of a coumarin or coumarin glycoside.

11. A method for reducing the infective ability or for inactivating bacterial pathogens by contacting said bacteria with a composition as claimed claim 1.

12. The method as claimed in claim 11 for use in food preparation.

13. The method as claimed in claim 11 for use in treatment of animals or humans infected with said pathogens.

14. The method as claimed in claim 11 for use in the disinfection of buildings or medical instruments.

15. The method as claimed in claim 11 wherein said bacterial pathogen is E. coli, Shigella spp., Salmonella spp., Listeria spp., or Straphylococcus spp.

16. The method as claimed in claim 15 wherein said bacterial pathogen is E. coli 0157.

17. The method as claimed in claim 15 wherein said bacterial pathogen is MRSA.

18. (canceled)
